(19) United States
(12) Patent Application Publication
(10) Pub. No.: US 2013/0029318 A1
(43) Pub. Date: Jan. 31, 2013

(54) Microchips and Methods for Testing a Fluid Sample

(71) Applicant: Yasuhisa Fujii, Kyoto (JP)

(72) Inventor: Yasuhisa Fujii, Kyoto (JP)

(73) Assignee: EMPIRE TECHNOLOGY DEVELOPMENT LLC, Wilmington, DE (US)

(21) Appl. No.: 13/322,761

(22) PCT Filed: Jul. 25, 2011
(86) PCT No.: PCT/US11/45234

§ 371 Date: Nov. 28, 2011

(51) Int. Cl. C12Q 1/70; G01N 1/00; G01N 33/566; G01N 33/542; C12M 1/34; G01N 11/00; G01N 33/573

(52) U.S. Cl. 435/5; 73/54.01; 73/64.56; 422/68.1; 422/82.05; 435/7.4; 435/7.9; 435/287.1; 435/287.2; 436/501

(57) ABSTRACT

Systems and methods for medical diagnosis or risk assessment for a patient are provided. A fluid-testing microchip is described which includes a filter compartment configured to receive a fluid sample from an inlet port, wherein the filter compartment comprises a plurality of beads coated with a defoaming agent, a micro-pump configured to transfer the fluid sample from the filter compartment to a test compartment, and the test compartment comprising a test component configured to test the fluid sample. The systems include an instrument for reading or evaluating the test data. These systems and methods are designed to be employed at the point of care, such as in emergency rooms and operating rooms, or in any situation in which a rapid and accurate result is desired.

### BACKGROUND

The following description is provided to assist the understanding of the reader. None of the information provided or references cited is admitted to be prior art.

Diagnostic tests performed in a laboratory and at the point-of-care (POC), are an integral part of the health care system. Such tests play an important role in all aspects of patient care, including disease-diagnosis, monitoring progression of therapy, as well as screening for health and infection. Molecular diagnostics tests (such as in vitro diagnostic (IVD) tests) are especially useful, as they pinpoint the exact cause of a particular clinical manifestation and thus help the physician to make a diagnosis and then prescribe the right treatment and therapy.

Current clinical laboratory tests frequently analyze whole blood or serum to check human health states. Clinical laboratory tests using blood include hematologic tests on blood morphology, coagulation-fibrinolysis system and leukocyte differentiation antigens; biochemical tests measuring proteins, enzymes, carbohydrates, electrolytes and drugs; internal secretion tests on various hormones and renin activity; immunological tests on tumor associated antigens, infectious disease antigens, autoantibodies and HLA; and genetic tests on chromosomes and oncogenes. The tests are performed by sampling several milliliters of blood from a patient and analyzing the sample with large automatic equipment in a testing center. Consequently, it may take several days to obtain the results. Blood sampling is associated with painful intrusion into the body, and is significantly burdensome, particularly to infants and senior people. Advances in point of care testing, represented by blood glucose monitoring tests for diabetic patients, is gradually increasing.

### SUMMARY

The present disclosure generally provides a fluid-testing microchip for point of care testing. In one aspect, the disclosure provides a fluid-testing microchip comprising: a filter compartment configured to receive a fluid sample from an inlet port, wherein the filter compartment comprises a plurality of beads coated with a defoaming agent; a micro pump configured to transfer the fluid sample from the filter compartment to a test compartment; and the test compartment comprising a test component configured to test the fluid sample. In one embodiment, the fluid sample is saliva.

In one embodiment, the test component is a piezoelectric oscillator configured to measure the viscosity of the fluid. In one embodiment, the piezoelectric oscillator comprises a first side and a second side, wherein the first side is coated with a silicone resin and is configured to be exposed to the fluid sample. In one embodiment, the test component includes a test reagent configured to react with an analyte in the fluid sample. In one embodiment, the test reagent is selected from the group consisting of: an antibody, an antigen, a pH indicator, or combinations thereof.

In one embodiment, the fluid-testing microchip further comprises a detector component that is configured to detect a reaction between the test reagent and the analyte in the fluid sample. In one embodiment, the detector component is a photodetector. In one embodiment, the detector component is a crystal oscillator.

In one embodiment, the fluid-testing microchip is made of glass. In one embodiment, the fluid-testing microchip further comprises a lid bonded to an upper surface of the filter compartment, wherein the lid is configured to contain the plurality of beads within the filter compartment. In one embodiment, the plurality of beads are of a size of about 0.2 μm to about 160 micro μm. In one embodiment, the defoaming agent is selected from the group consisting of: a silicon-type defoaming agent, a surfactant, a polyether, a higher alcohol, or combinations thereof.

In one embodiment, the micro pump is selected from the group consisting of: a volume-changing micro pump, a diffuser type mechanical micro pump, an electroosmotic flow micropump, a centrifugal pump, a syringe pump, a plunger pump, or combinations thereof.

In one aspect, the present disclosure provides a method for testing fluid, the method comprising: passing a fluid sample through microbeads coated with a defoaming agent; pumping the defoamed fluid into a test compartment; and testing the defoamed fluid.

In one embodiment, testing the defoamed fluid comprises measuring the viscosity of the defoamed salvia. In one embodiment, testing the defoamed fluid comprises reacting a test reagent with an analyte in the defoamed fluid. In one embodiment, the method further comprises detecting a reaction between the test reagent and the analyte.

In another aspect, the present disclosure provides a system for testing fluid comprising: a filter component configured to defoam a fluid sample; a micro pump; a test component configured to test the defoamed fluid; and a housing unit configured to provide power to the micro pump, wherein the system is configured to indicate a result of the test to a user.

In one embodiment, the test component measures the viscosity of the defoamed fluid. In one embodiment, the test component comprises reagent that reacts with an analyte in the defoamed fluid.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1 is an overhead view of an illustrative embodiment of a fluid-testing microchip.

FIGS. 2A and 2B are cross-section views of illustrative embodiments of a fluid-testing microchip.

FIG. 3 is an overhead view of a crystal oscillator sensor used in an illustrative embodiment of a fluid-testing microchip.

FIG. 4 is a perspective view of a housing unit used in conjunction with an illustrative embodiment of a fluid-testing microchip.

FIG. 5 is a flow diagram depicting operations performed in an illustrative embodiment.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure.

Systems and methods for medical diagnosis or risk assessment for a patient are provided. These systems and methods are designed to be employed at the point of care, such as in clinics, emergency rooms, operating rooms, hospital laboratories and other clinical laboratories, doctor's offices, in the field, or in any situation in which a rapid and accurate result is desired. The systems and methods process fluid samples from a patient using diagnostic tests or assays, including immunoassays, chemical assays, calorimetric assays, fluorometric assays, chemiluminescent and bioluminescent assays, and other such tests, and provide an indication of a medical condition or risk or absence thereof. The patient can be a human patient or a non-human patient. The patient can be a mammal or other animal. The patient can be a non-human animal such as dog, cat, horse, cow, pig, goat, monkey, elephant, giraffe, rhinoceros, bear, moose, snake, alligator, and so on.

In one aspect, this disclosure relates to a saliva testing device which can test the saliva of a subject for disease or other medical condition. Saliva contains almost all the clinical ingredients in blood, although in lower concentrations. Therefore, an assay using saliva can be used to diagnose disease, check for drug use, etc., in a manner similar to blood may be used. Unlike other forms of fluid specimens, such as blood or urine, collection of saliva for diagnostic purposes may be complicated by many factors, such as the low volumes of salivary fluid secreted into the oral cavity, the relatively high viscosity thereof, and the diverse anatomic dispersion of the salivary glands. Most techniques for collection of saliva involve the use of capillary tubes, suction into micropipettes, chewing on paraffin or foam, and/or aspiration from the mouth into polypropylene syringes. By contrast, in some embodiments, the saliva testing devices described herein provide for the direct testing of a saliva sample without the need for pre-processing the saliva sample.

The fluid-testing microchips may be used in a variety of contexts. Check-ups for periodontal disease and tooth decay may include sampling saliva in place of blood. Testing methods to detect measles, epidemic parotitis (mumps), German measles, hepatitis (types A, B and C), breast cancer, Alzheimer's disease and cystic fibrosis by sampling saliva instead of blood have been developed, and an HIV test using saliva was approved by the US Food and Drug Administration (FDA) in 2004. However, the saliva tests that have been developed to date require large apparatuses, and can be performed only at some specialized hospitals.

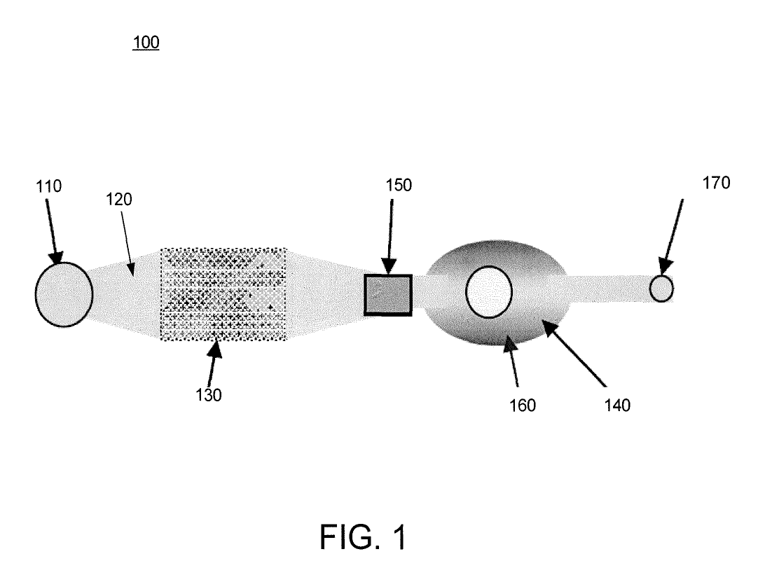

FIG. 1 is an overhead view of an illustrative embodiment of a fluid-testing microchip 100. In one illustrative embodiment, the fluid-testing microchip 100 may be used to test a saliva sample. The fluid-testing microchip 100 has an inlet port 110 for receiving the saliva sample. In some embodiments, the saliva sample may be diluted with water, saline, or another solvent prior to being introduced to the inlet port 110. As saliva can be highly viscous, the fluid-testing microchip 100 is made of glass, plastic, or other inert material that does not interfere with the assay procedure. In some embodiments the glass, plastic, or other inter material may be coated with a hydrophilic.

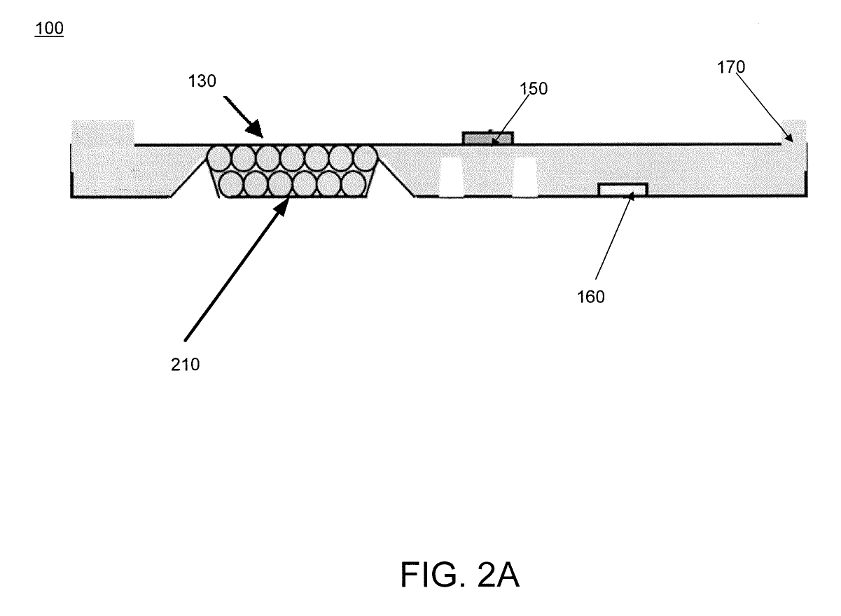

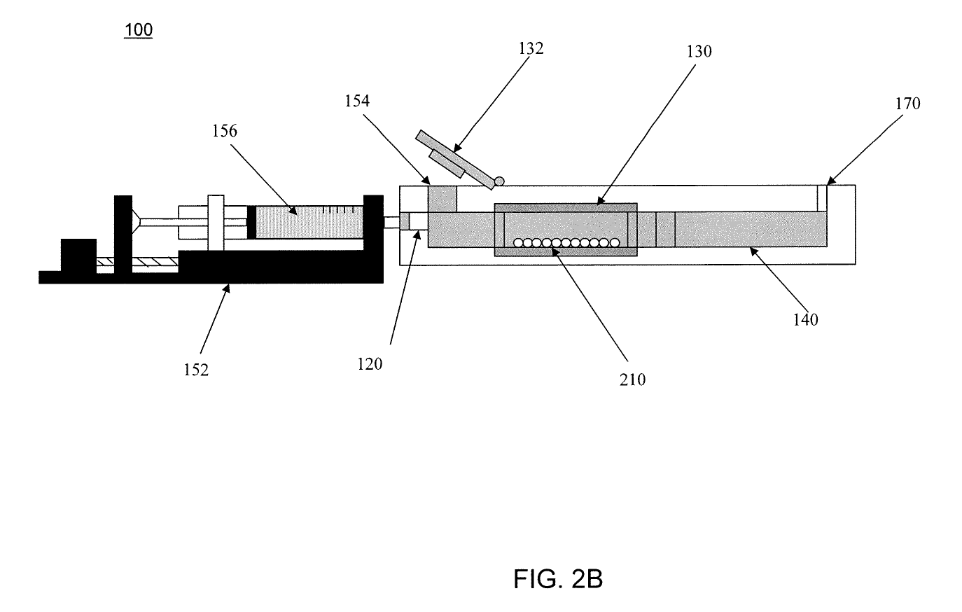

The saliva sample travels via a portion of a flow channel 120 to a filter compartment 130. FIG. 2A is a cross section view of an illustrative embodiment of the fluid-testing microchip 100. The filter compartment 130 includes a plurality of beads 210 coated with a defoaming agent. Various defoaming agents can be used, including but not limited to, a silicon-type defoaming agent, a surfactant, a polyether, a higher alcohol, or combinations thereof. A silicone-type defoaming agent may be used for both aqueous and non-aqueous foaming solutions. Specific, but non-limiting, examples of a silicone-type defoaming agent include FS Antifoam DB-110N, FS Antifoam 91, or Toray SH 5561 Emulsion. Organic defoaming agents may also be used for aqueous foaming solutions. The plurality of beads 210 are retained in the filter compartment 130 through a lid (not shown). The lid can be made of the same material as the fluid-testing microchip 100, for example Pyrex glass, and may be attached or bonded to the fluid-testing microchip 100 using fluid glass.

The plurality of beads 210 are configured to defoam a fluid, such as saliva, and additionally, trap impurities within the fluid. In one configuration, the plurality of beads 210 are spherical in shape. In this configuration, spaces between the plurality of spherical beads trap foams and impurities. For example, the plurality of beads 210 can trap food particles, plaque, and bacteria. Removing foam and impurities can help to improve the accuracy of tests on the fluid. Different sized beads may be used depending on the configuration of the fluid-testing microchip 100. Sizes of beads which may be used in the fluid-testing microchip 100 include, but are not limited to, about 0.2 micrometers (μm), about 2 μm, about 20 μm, about 60 μm, about 100 μm, and about 140 μm, and ranges between any two of these values. In other embodiments, the beads are sized from about 0.1 μm to about 500 μm. Different sized beads may be used simultaneously in the fluid-testing microchip 100. Individual beads may be composed of, but not limited to, silica or a copolymer resin. The copolymer resin may be a resin of polymer of polyacrylamide and agarose or a high hydropholic copolymer resin. Specific non-limiting examples of beads that may be used in the fluid-testing microchip 100 include Ultrogel® AcA (Pall Corporation), Trisacryl® GF05M (Pall Corporation), or UNK HIPRESICA (Ube Nitto Kasei Co., Ltd.).

In one embodiment, the defoamed fluid is transferred from the filter compartment 130 to a test compartment 140 using a micro pump 150. In one configuration, the micro pump 150 is a volume-changing micro pump. Other varieties of micro pumps can be used, including but not limited to, a diffuser type mechanical micro pump, an electroosmotic flow micro pump, a centrifugal pump, a syringe pump, a plunger pump, or combinations thereof, known in the art. FIG. 2B illustrates another embodiment, where a syringe pump 152 is used instead of the micro pump. A syringe pump is operably connected to the flow channel 120. Fluid 156 in the syringe can be injected into the microchip through the flow channel 120. A microchip can include an overflow outlet port 154 that handles any overflow liquid as the liquid is moved through the microchip. The filter compartment 130 can have a lid 132 that retains the plurality of beads 210 within the filter compartment 130. The lid 132 can be made of the same material as the fluid-testing microchip 100, for example Pyrex glass, and may be attached or bonded to the fluid-testing microchip 100 using fluid glass. In one embodiment, the lid 132 can be hinged allowing the lid 132 to open and close.

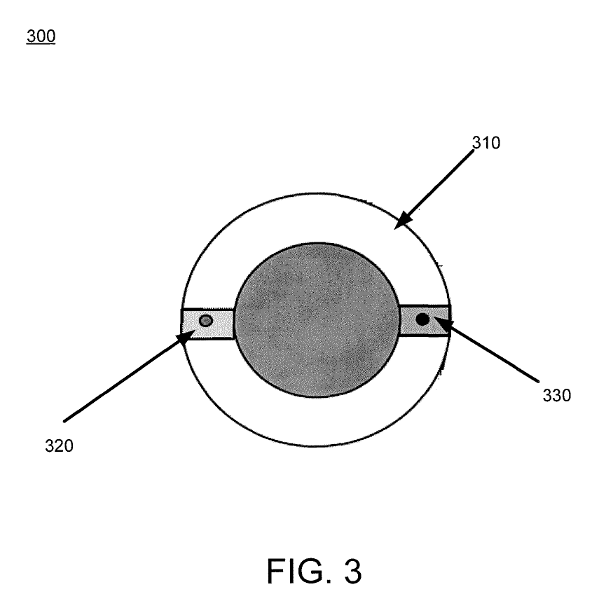

The test compartment 140 can include a test component 160. In one embodiment, the test component 160 includes a piezoelectric oscillator. FIG. 3 is an overhead view of a crystal oscillator sensor 300 used in an illustrative embodiment of the fluid-testing microchip 100. The crystal oscillator sensor 300 can have a crystal 310, a rear electrode 320, and a front electrode 330. In one embodiment, the rear electrode 320 and the front electrode 330 are made of a 150 nanometer (nm) layer of gold. Other materials can be used, such as, but not limited to, copper. Different thicknesses of materials may also be used, such as, but not limited to, 100 nm, 200 nm, 1 μm, 50 μm, and ranges between any two of these values. The crystal oscillator sensor 300 also includes a first side that is coated with a silicone resin and is configured to come into contact with the fluid sample.

In one illustrative embodiment, the piezoelectric oscillator can be configured to test the viscosity of a saliva sample. The change in resonance frequency of quartz crystal microbalance due to a change in viscosity of a fluid is calculated in the formula:

$$Δ f = - {{f}_{o}^{\frac{3}{2}} ( η l \times ρ l \div π \times {ρ}_{q} {μ}_{q} )}^{\frac{1}{2}} ,$$

where Δf is the change in frequency, f₀ is the resonance frequency, ηl is the fluid viscosity, ρl is the fluid density, ρ_{q} is the density of crystal and μ_{q} is the frequency constant. The density of quartz crystal is

$$2.65 \frac{g}{c}$$

and the frequency constant is 1.65×10⁵ Hzcm. Change in the resonance frequency is, therefore, proportional to the square root of the fluid density multiplied by the fluid viscosity. As the density of saliva typically remains constant, change in viscosity is calculated from the change in resonance frequency. Various medical conditions affect the viscosity of saliva. For instance, alveolar pyorrhea, tooth decay, sympathetic activity, and parasympathetic activity can affect the viscosity of saliva. The test compartment 160 can also be configured to test other properties of a fluid sample, such as, but not limited to measuring the hydration of the sample, detecting certain biomarkers or microbes in the sample, and reacting a test reagent with the sample. In another embodiment, the fluid-testing microchip 100 can be configured to test the sample using two or more of the tests simultaneously.

In another illustrative embodiment, the test compartment 140 can include a dried test reagent that is immobilized on the bottom of the test compartment 140. The fluid sample containing or suspected to contain an analyte enters the test compartment and reacts with the test reagent. Various test reagents can be used, including but not limited to, an antibody, an antigen, a pH indicator, or combinations thereof. A detector component (not shown) detects the reaction between the test reagent and the analyte. Detector components include, but are not limited to, a crystal oscillator or a photodetector. As an illustrative example, a photodetector is used to measure turbidity of the fluid sample. Fluid that has been tested continues through the test compartment 140 to the vent 170. Upon reaching the vent 170, the fluid exits the fluid-testing microchip 100.

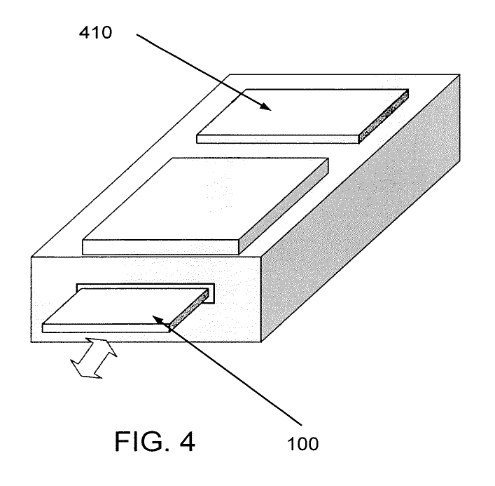

FIG. 4 is a perspective view of an illustrative housing unit 400 used in conjunction with an illustrative embodiment of the fluid-testing microchip 100. The fluid-testing microchip 100 is inserted into a housing unit 400, which supplies power to the micro pump 150 and the optional detector component. The micro pump 150 controls the flow of the fluid sample through one or more or all of the flow channels 120 through inlet port 110, the filter compartment 130, the test compartment 140 and finally to the vent 170. The housing unit 400 can also include a result indication component 410, which can be, but is not limited to, a display screen, LED, or light.

In an illustrative embodiment, the fluid-testing microchip 100 can be made of Pyrex glass. To construct the fluid-testing microchip 100, Pyrex glass may be subjected to a two-stage etching by wet etching with hydrofluoric acid or reactive ion etching. The filter compartment 130 and outlet area of the micro pump may be shallowly etched. The other areas of the fluid-testing microchip 100 may be deeply etched. The plurality of beads 210 may be inserted into the filter compartment 130, which is then covered with a thin plate of Pyrex glass. The thin plate of Pyrex glass may be attached to the fluid-testing microchip 100 using fluid glass. A piezoelectric element of a predetermined size may be bonded onto the diaphragm part of the micro pump 150. The piezoelectric element may be between about 0.5 mm and about 6 mm in length, about 0.1 mm and about 2 mm in width, and about 0.02 mm and about 0.5 mm in depth.

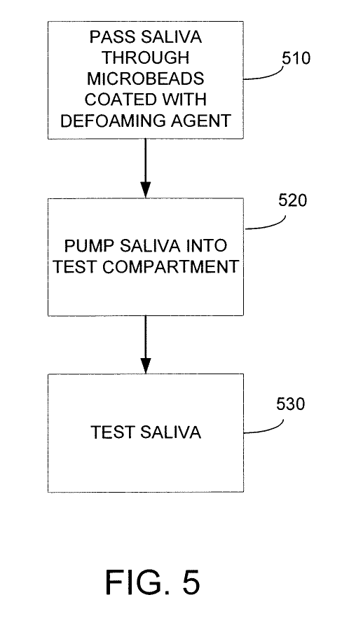

FIG. 5 is a flow diagram depicting operations performed in an illustrative embodiment for testing a saliva sample. Additional, fewer, or different operations may be performed depending on the particular embodiment. A saliva specimen may be extracted from or obtained from a subject. In an operation 510, a saliva sample passes through the plurality of microbeads 210 that are coated with a defoaming agent. Passing the saliva sample through the plurality of microbeads defoams the saliva sample and also filters impurities. In an operation 510, the saliva sample is pumped into the test compartment 140. In an operation 530, the saliva sample is tested using the test component 160. Illustrative but non-limiting examples of tests include measuring the viscosity of the saliva sample, the hydration of the saliva sample, detecting certain biomarkers or microbes, and reacting a test reagent with the saliva sample. A detector can be used to detect the reaction of the test reagent with the saliva sample. In another embodiment, the saliva sample can be tested using two or more of the tests simultaneously.

Any assay is intended for use in the systems and methods herein. Such assays include, but are not limited to: any assay that relies on colorimetric or spectrometric detection, including fluorometric, luminescent detection, such as creatine, hemoglobin, lipids, ionic assays, and blood chemistry. Any test that produces a signal, or from which a signal can be generated, that can be detected by a detector, such as a photodetector, is intended for use as part of the systems provided herein.

Immunoassays, including competitive and non-competitive immunoassays, are among those suitable for determination of the presence or amount of analyte in a patient saliva sample. A number of different types of immunoassays are well known using a variety of protocols and labels. Immunoassays may be homogeneous, i.e. performed in a single phase, or heterogeneous, where antigen or antibody is linked to an insoluble solid support upon which the assay is performed. Sandwich or competitive assays may be performed. The reaction steps may be performed simultaneously or sequentially. Any known immunoassay procedure, particularly those that can be adapted for use in combination with lateral flow devices as described herein, can be used in the systems and methods.

Any antibody, including polyclonal or monoclonal antibodies, or any fragment thereof, such as the Fab fragment, that binds the analyte of interest, is contemplated for use herein. For example, a mouse monoclonal antibody may be used in a labeled antibody-conjugate for detecting an analyte. Alternatively, a polyclonal anti-Ig antibody may also be used to bind a primary antibody to form a sandwich complex.

In one embodiment, an antibody conjugate containing a detectable label may be used to bind the analyte of interest. The detectable label used in the antibody conjugate may be any physical or chemical label capable of being detected on a solid support using a reader, for example, a reflectance reader, and capable of being used to distinguish the reagents to be detected from other compounds and materials in the assay. Suitable antibody labels are well known to those of skill in the art. The labels include, but are not limited to, enzyme-substrate combinations that produce color upon reaction, colored particles, such as latex particles, colloidal metal or metal or carbon sol labels, fluorescent labels, and liposome or polymer sacs, which are detected due to aggregation of the label. An illustrative label is a colored latex particle. In an alternative embodiment, colloidal gold is used in the labeled antibody conjugate.

Any analyte that can be detected in an assay, particularly colorimetric assays, including immunoassays, is associated with a disorder is contemplated for use as a target herein. Suitable analytes are any which can be used, along with a specific binding partner, such as an antibody, or a competitor, such as an analog, in an assay. Analytes may include, but are not limited to proteins, haptens, immunoglobulins, enzymes, hormones (e.g., hCG, LH, E-3-G estrone-3-glucuronide and P-3-G (progestrone-3-glucuronide)), polynucleotides, steroids, lipoproteins, drugs, bacterial or viral antigens, such as Streptococcus, Neisseria and Chlamydia, lymphokines, cytokines, and the like.

In conducting the assay, a patient saliva sample is obtained or provided. The saliva sample may include fluid and particulate solids, and, thus, can be filtered prior to application to the assay microchip. A volume of the test sample is delivered to the microchip using any known means for transporting a biological sample, for example, a standard plastic pipet. In one embodiment, an analyte in the sample binds to the labeled reagent, and the resulting complex migrates along the test strip. Alternatively, the sample may be pre-mixed with the labeled conjugate prior to applying the mixture to the test strip. When the labeled antibody-analyte complex encounters a detection zone of the microchip, the immobilized antibody therein binds the complex to form a sandwich complex, thereby forming a colored stripe.

The results of the assays can be determined in a variety of ways, including visual inspection of the microchip. In other instances, instrumentation such as reflectance and other readers, including densitometers and transmittance readers, may be used. Any reader that upon combination with appropriate software can be used to detect images and digitize images particularly bar codes or the lines and stripes produced on chromatographic immunoassay devices or on gels or photographic images thereof, such as the lines on DNA and RNA sequencing gels, X-rays, electrocardiograms, and other such data, is intended for use herein.

In an illustrative embodiment, a sample is applied to a fluid-testing microchip, and colored or dark bands are produced. The intensity of the color reflected by the colored label in the test region (or detection zone) of the test strip is, for concentration ranges of interest, directly proportional or otherwise correlated with an amount of analyte present in the sample being tested. The color intensity produced may be read using a reader device, for example, a reflectance reader, adapted to read the microchip. The intensity of the color reflected by the colored label in the test region (or detection zone) of the microchip is directly proportional to the amount of analyte present in the sample being tested. In other words, a darker colored line in the test region indicates a greater amount of analyte, whereas a lighter colored line in the test region indicates a smaller amount of analyte. The color intensity produced, i.e., the darkness or lightness of the colored line, is read using a reader device, for example, a reflectance reader, adapted to read the test strip. A reflectance measurement obtained by the reader device may be correlated to the presence and/or quantity of analyte present in the sample. The system may also correlate such data with the presence of a disorder, condition or risk thereof.

Optionally, in addition to reading the test strip, the reader may be adapted to read a symbology, such as a bar code, which is present on the test strip or housing and encodes information relating to the test strip device and/or test result and/or patient, and/or reagent or other desired information. Typically, the associated information is stored in a remote computer database, but can be manually stored. In other embodiments, the symbology can be imprinted when the device is used and the information encoded therein.

### EXAMPLES

The present compositions and methods will be understood more readily by reference to the following examples, which are provided by way of illustration and are not intended to be limiting in any way.

### Example 1

### Construction of the Microchip

A 35 millimeter (mm)×20 mm×0.5 mm piece of Pyrex glass is subjected to a wet etching processes using hydrofluoric acid and a dry etching process using Deep Reactive Ion Etching (DRIE) apparatus with CF₄ gases. The filter compartment and test compartment are etched to a depth between 100 and 300 micrometers (m). The flow channel, inlet port, vent, and the inlet and outlet are of the micro pump are etched to a depth between 20 μm and 50 μm. Ultrogel® AcA 44 beads are coated with FS Antifoam DB-110N defoaming agent. The filter compartment is filed with the coated beads. A 35 mm×20 mm×0.5 mm lid made of Pyrex glass is bonded using fluid glass over the filter compartment. A micro pump can be made from a piezoelectric oscillator that is bonded onto the Pyrex glass, with its inlet connected to the filter compartment via the flow channel, and its outlet connected to the test compartment via the flow channel. A crystal oscillator is bonded on the bottom of test compartment.

### Example 2

### Testing for Alveolar Pyorrhea

A 0.2 milliliter (ml) sample of saliva is diluted with 0.8 ml of water. The diluted saliva sample is introduced into the inlet port of the fluid-testing microchip and through a portion of the flow channel to the filter component. The filter component is filed with Ultrogel® AcA 34 beads. A syringe pump or micro pump moves the diluted saliva through the filter component and into a test compartment. The test compartment includes one or more components to measure the viscosity of defoamed saliva.

### Example 3

### Testing for Tooth Decay

A 0.2 ml sample of saliva is diluted with 0.8 ml of saline. The diluted saliva sample is introduced into the inlet port of the fluid-testing microchip and through a portion of the flow channel to the filter component. The filter component is filed with UNK HIPRESICA beads of size 10 μm. A syringe pump or micro pump moves the diluted saliva through the filter component and into a test compartment. The test compartment includes one or more components that count Streptococcus mutans and Actobacillus.

One or more flow diagrams have been used herein. The use of flow diagrams is not meant to be limiting with respect to the order of operations performed. The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

The term “about” and the use of ranges in general, whether or not qualified by the term about, means that the number comprehended is not limited to the exact number set forth herein, and is intended to refer to ranges substantially within the quoted range while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

What is claimed is:

1. A fluid-testing microchip comprising:
a filter compartment configured to receive a fluid sample from an inlet port, wherein the filter compartment comprises a plurality of beads coated with a defoamine agent;
a micro pump configured to transfer the fluid sample from the filter compartment to a test compartment; and
the test compartment comprising a test component configured to test the fluid sample.

2. The fluid-testing microchip of claim 1, wherein the fluid is saliva.

3. The fluid-testing microchip of claim 1, wherein the test component is a piezoelectric oscillator configured to measure the viscosity of the fluid.

4. The fluid-testing microchip of claim 3, wherein the piezoelectric oscillator comprises a first side and a second side, wherein the first side is coated with a silicone resin and is configured to be exposed to the fluid sample.

5. The fluid-testing microchip of claim 1, wherein the test component includes a test reagent that is configured to react with an analyte in the fluid sample.

6. The fluid-testing microchip of claim 4, wherein the test reagent is selected from the group consisting of: an antibody, an antigen, a pH indicator, or combinations thereof.

7. The fluid-testing microchip of claim 4 further comprising a detector component that is configured to detect a reaction between the test reagent and the analyte in the fluid sample.

8. The fluid-testing microchip of claim 7, wherein the detector component is a photodetector.

9. The fluid-testing microchip of claim 7, wherein the detector component is a crystal oscillator.

10. The fluid-testing microchip of claim 1, wherein the fluid-testing microchip is comprised of glass.

11. The fluid-testing microchip of claim 1, further comprising a lid bonded to an upper surface of the filter compartment, wherein the lid is configured to contain the plurality of beads within the filter compartment.

12. The fluid-testing microchip of claim 1, wherein the plurality of //beads are of a size of about 0.2 μm to about 160 μm.

13. The fluid-testing microchip of claim 1, wherein the defoaming agent is a silicon-type defoaming agent, a surfactant, a polyether, a higher alcohol, or combinations thereof.

14. The fluid-testing microchip of claim 1, wherein the micro pump is a volume-changing micro pump, a diffuser type mechanical micro pump, an electroosmotic flow micro pump, a centrifugal pump, a syringe pump, a plunger pump, or combinations thereof.

15. The fluid-testing microchip of claim 1, wherein the fluid sample is diluted fluid.

16. A method for testing fluid, the method comprising:
providing a fluid sample;
passing the fluid sample through microbeads coated with a defoaming agent to form a defoamed fluid;
pumping the defoamed fluid into a test compartment; and
testing the defoamed -fluid.

17. The method of claim 16, wherein the fluid is saliva.

18. The method of claim 16, wherein testing the defoamed fluid comprises measuring the viscosity of the defoamed salvia.

19. The method of claim 16, wherein testing the defoamed fluid comprises reacting a test reagent with an analyte in the defoamed fluid.

20. The method of claim 19, further comprising detecting the reaction between the test reagent and the analyte.

21. The method of claim 16, wherein the fluid sample comprises diluted fluid.

22.-28. (canceled)
